(19) United States
(12) Patent Application Publication
(10) Pub. No.: US 2017/0258410 A1
(43) Pub. Date: Sep. 14, 2017

(54) METHOD AND APPARATUS FOR PREDICTION OF EPILEPTIC SEIZURES

(71) Applicant: UNIVERSITY OF WINDSOR, Windsor (CA)

(72) Inventor: Robin GRAS, Windsor (CA)

(73) Assignee: UNIVERSITY OF WINDSOR, Windsor (CA)

(21) Appl. No.: 15/506,704

(22) PCT Filed: Aug. 26, 2015
(86) PCT No.: PCT/CA2015/000476

§ 371 Date: Feb. 24, 2017

Related U.S. Application Data
(60) Provisional application No. 62/042,535, filed on Aug. 27, 2014.

(51) Int. Cl. A61B 5/00; A61B 5/04; A61B 5/0476

(52) U.S. Cl. CPC A61B 5/746; A61B 5/0476; A61B 5/0022; A61B 5/4094; A61B 5/04012; A61B 5/7253

(57) ABSTRACT

A system for predicting epileptic seizures includes sensors operable to record a wearer's brain activity. The sensors electronically communicate with a processor configured to receive and store output EEG oscillations and activities. A threshold electrical fluctuation level is identified as the level electrical activity experienced at the onset of a seizure event, and is then stored in the PDA memory as a predetermined threshold value. The processor analyzes the input EEG data logged for a recording period, and the logged data is broken into a number of data values across a series of individual set sampling periods. Convert collected data value readings for individual sampling periods as a non-linear measure value using fractal dimension, P&H and/or Lyapunov weighing. The calculated values for a predicted next time intervals extending the sampling period is projected forward and compared against the predetermined threshold value to indicate a likely seizure event.

### RELATED APPLICATIONS

This application claims priority to and benefit of 35 USC §119(e) of U.S. Provisional Patent Application Ser. No. 62/042,535, filed 27 Aug. 2014, the disclosure of which is hereby incorporated herein by reference in its entirety.

### SCOPE OF THE INVENTION

The present invention relates to a method and system for performing predictive modeling on more complex data, and particularly a system for achieving the predictive chaos analysis of non-linear data or events, and more preferably a system and method for analysis of EEG readings used to indicate the likely onset of epileptic seizures. More preferably, EEG readings data used to predict epileptic seizures are subjected to a further transformation to provide a model which is operable to predict or forecast the likely occurrence of an epileptic seizure that is about to occur in the future.

### BACKGROUND OF THE INVENTION

It has been recognized that long-term time series prediction has promise for many applications, such as prediction of earthquakes, financial market prediction, and the like, and where non-linear properties of a time series are evaluated and used for long-term prediction.

The prediction of complex time series future values is therefore a major concern for scientists with applications in various fields of science. Many natural phenomena such as variations in population, the orbit of astronomical objects and earth's seismic waves could be subject to a prediction algorithm. Prediction also has application in forecasting economic time series. Time series analysis of earth's seismic waves can be used for earthquake prediction. Prediction of other data such as population projections may be used to predict species extinction before they reach a tipping point may provide another application of time series prediction.

It has been shown that many data generated by such natural phenomena follow chaotic behavior. Various authors have proposed models for the predictive analysis of non-linear and chaotic events. Clements et al. in Forecasting Economic and Financial Time-Series with Non-linear Models, International Journal of Forecasting 20 (2004) 169-183 highlights the difficulties associated with conventional non-linear models used in the prediction of economic behavior and performance. Further, Yang et al. in Forecasting the Future: Is It Possible for Adiabatically Time-Varying Non-Linear Dynamical Systems? CHAOS 22, 033119 (2012) proposes a non-linear dynamical system in which parameters vary adiabatically with time where measured time series is used to predict future asymptotic attractors to the system. Wang et al. in Fuzzy Prediction of Chaotic Time Series Based on Fuzzy Clustering, Asian Journal of Control Vol. 13, No. 4, pp 576-581 (2011) also describes a process for time series prediction for use in weather forecasting, speech coding, noise cancellation and the like.

Most of the existing methods for complex time series prediction are based on modeling the time series to predict future values, although there are other types of methods like agent-based simulation that model the system generating the time series [Filippo Neri: Learning and Predicting Financial Time Series by Combining Natural Computation and Agent Simulation. Evo Applications (2) 2011: 111-119]. The model based approaches may be mainly classified in two main domains: linear models like ARIMA (AutoRegressive Integrated Moving Average) [G. Box and G. Jenkins, Time Series Analysis: Forecasting and Control, Holden-Day, San Francisco, 1976] and non-linear models like MLP [Zirilli, J.: Financial prediction using Neural Networks. International Thompson Computer Press (1997)] and GARCH [Bollerslev, Tim (1986). “Generalized Autoregressive Conditional Heteroskedasticity”, Journal of Econometrics, 31:307-327]. However, studies have concluded that there was no clear evidence in favor of non-linear over linear models in terms of forecast performance.

Although chaotic behaviors are deterministic, their complex properties make them hard to be distinguishing from random behavior. They are well known to be strongly dependent on initial conditions, small changes in initial conditions possibly leading to tremendous changes in subsequent time steps, and particularly difficult to predict. Since the exact conditions for many natural phenomena are not known and the properties of a chaotic time series are very complex, previously it is has proven difficult to model these systems.

Heretofore, however, there has been no robust procedure that can estimate an accurate model for chaotic time series. For conventional predictive methods, the prediction error increase dramatically with the number of time points predicted. As a result, most of existing methods focus on very short-term prediction to reach a reasonable level of accuracy. For example, for financial time series prediction a simple step ahead, may not prove overly helpful for acting against financial recession beforehand. Despite of the difficulties inherent to non-linear modeling, non-linear analysis has the potential for a variety of commercial applications.

### SUMMARY OF THE INVENTION

The applicant has recognized that the prediction of non-linear health events provides significant health and/or social benefits. In medical science there are many applications for which an efficient prediction algorithm could save lives. By example, a large number of time series gained from the human body can be used as an origin of the decision making process to treat or prevent dangerous diseases such as heart attacks, cancers, epilepsy and Alzheimer's. By way of example, individuals who suffer from epilepsy may be prone to severe and unexpected seizures, which may prevent epileptics from performing routine tasks such as driving or operating heavy equipment, and which otherwise have the potential to result in the individual's injury should the onset of a seizure occur where the individual is physically in a vulnerable location, such as a stairwell or bathtub. It is recognized that by being able to predict the likely future onset of a seizure, an epileptic may be given advanced warning to provide for sufficient time to prepare for the seizure onset, and relocate to a physically safe environment.

The present invention provides a method and system for generating predictive models, and more particularly for undertaking the predictive analysis of non-linear historical data and/or real-time data to predict the likelihood of a selected event or future trend. More preferably, with the present invention, an appropriate method and system may be used in predicting long term variations across a variety of technologies, including without restriction, the prediction of medical events, the prediction of seismological or meteorological events or outcomes; the prediction of ecosystem trends; the prediction of health, pandemic and/or demographic events; as well as other macrogeographic events.

The present invention seeks to provide a simplified process and system for predictive analysis, and more particularly which may provide improved reliability for chaotic or event predictions.

In one non-limiting embodiment, the invention provides a system and method for time series prediction which analyzes of continuous electroencephalography (EEG) data. The system operates to output a user or other professional a signal or display indicating where in a further time series an epileptic seizure will occur. More preferably, the system operates whereby a predicted seizure which is later confirmed by recorded EEG data records showing the epileptic seizure to have occurred is updated, and used to establish future or a next predicted seizure event.

In a preferred construction, the invention provides a system for predicting an epileptic seizure which includes one or more EEG sensors for positioning on a user or subject's skull. The EEG sensors are operable to continuously record the user's brain electrical activity either continuously or over a selected period of time, and preferably reading fluctuations and output the user's EEG data. Most preferably, the sensors are adapted for electronic, and preferably wireless communication with a processor, which for example may be in the form of a device with computational capability such as a computer, tablet, smart phone, smart watch, personal digital assistant (PDA) or the like (hereinafter collectively “a personal digital assistant or PDA”). The PDA is configured to receive and process the output EEG readings and activities.

In undertaking the EEG recording, a threshold value in the processed data output of the computational device is determined as the level/threshold where the user's brain will experience at or about the onset of an epileptic seizure event. The threshold value is then stored in the PDA memory as a predetermined threshold value, which is indicative of an epileptic-seizure event. Alternately, the predetermined threshold value may be calculated and/or input independently, as for example by averaging empirical data collected from a number of third party subjects, and prestored in the PDA memory as part of a software component.

The PDA further operates to analyze the input EEG data received from the sensors. To initiate the system, initial EEG data is recorded continuously for a period of between 0.5 to 5 hours, and preferably about 60 minutes. The initial EEG data is preferably obtained directly from the user and used as a baseline upon which data processing may commence to produce reference values and start predicting values. EEG data is preferably logged and processed by the PDA in time interval ranging from 10 to 360 minutes, and typically between about 20 to 90 minutes, and most preferably about 60 minutes. Most preferably, the baseline recording is continuous.

In an exemplary mode, the system operates to output a warning to the user at least 16 minutes prior to the time the user is anticipated to experience the onset of an epileptic event such as a grand mal or Tonic-conic seizure.

In a preferred mode of operation the continuous data streaming into the computational device the device takes an EEG value every 10 to 60 seconds, and preferably about every 20 seconds. At the end of the 60 minutes of baseline data recording and in a preferred mode, the collection of 180 EEG data point an initial time series “S_{N}”(x₁, x₂, . . . x_{N}) is provided. The system then commences processing data and predicting values into the future. For the purpose of data analysis, a time window (L) of 10 to 60 minutes, and preferably 20 minutes is selected. The 20 second sampling interval selected; the 60 minute baseline selected and the 20 minute time window (L) are preferred and may be varied based on the input EEG data, the base data being used and the degree of accuracy to be achieved.

The processor is operated to convert or transform the collected EEG data value readings for individual time series over set sampling periods (x₁, x₂, . . . x_{N}) to “V” value for selected time intervals of the recording period “L”. Most preferably, the processor transforms or converts the initial data set or series (S_{N}) over the selected interval period (L) into a new data series S^{m}_{N} to calculate to a single value V(Sᵢ) using a Lyapunov characteristic exponent, and/or Fractional Dimension and/or P&H (“Poincaré Section and Higuchi Fractal Dimension”) methods. This may obtain a quantification of the rate of separation of events and/or fractal deviation to provide a statistical index of the complexity of the data over the selected time interval.

The “V” value is a measure of chaos in the data set (x₁, x₂, . . . x_{L},) and is plotted as “y_{L}” the first point of the transformed time series at time position “L”. The window is moved one data point to the right (x₂, x₃, . . . x_{L+1},), and the “V” value is calculated and plotted as “y_{L+1}” at time sequence position L+1. This is repeated until all data points from time position L to N have been converted.

The present invention provides in another non-limiting embodiment, a simplified processes and systems for predictive analysis, and more particularly which may provide improved reliability for chaotic or event prediction. More preferably, the invention provides a system and method for time series prediction which analyzes of continuous electroencephalography (EEG) data and can predict where in the future time series an epileptic seizure will likely occur, and which may be later confirmed by the recorded EEG data showing epileptic seizures.

In another non-limiting embodiment, the invention provides a system for predicting an epileptic seizure which includes one or more EEG sensors for positioning on a subject's skull, and which are operable to continuously record the wearer's brain electrical wave activity, and preferably electrical reading fluctuations and output the user's EEG data. The system may for example, be provided with wearable electronics and/or diagnostics such as a smart watch or glasses, sensor bands, and/or EEG sensors held to skin with electrically conductive adhesive. Most preferably, the sensors are adapted for wireless electronic communication with a processor or PDA. The PDA is configured to receive, store and process data representative of the output EEG oscillations and activity. In undertaking the EEG recording, a user-specific threshold value is preferably determined based on the processed continuous EEG signal over a period of time, and which identifies a level of the user's brain electrical activity above which the user experiences or is about to experience the onset of a selected epileptic seizure event. This threshold level value is then stored in the PDA memory as a predetermined threshold value, which is indicative of an epileptic seizure occurrence.

The PDA processor operates to analyze the input EEG data. To initiate the system, EEG data is preferably continuously recorded for at least 40 minutes, and more preferably about 60 minutes±15 to obtain directly from the user the baseline upon which data transformation is done. The transformation produces a new series of values upon which data processing may commence to produce reference values and provide predictive EEG data derived values into the future. The predicated data values are then preferably logged into the system; and are themselves then transformed; and processed by the PDA continuously for a recording period ranging from as little as 60 to 240 minutes; to days; weeks or monthly or semi-annual periods. The data analysis window is typically between about 15 to 30 minutes, and more preferably 20 minutes. The selection of the data processing window is selected based on data characteristics and the objective of the prediction. The level of confidence in the predicted data decreases as the predicted data window gets larger and also with data that is predicted into the future beyond the time interval of the processing window. Most preferably, the recording period continues through uninterrupted after the baseline data has been recorded. The system operates to output a warning to the user 5 to 120 minutes, and preferably at least 16 minutes prior to the time the user is expected to experience an epileptic event, such as a grand mal or Tonic-conic seizure.

In accordance with another possible preferred mode of operation, a selected number of data points N of the non-linear variable are monitored over a selected time sampling period T(_{LN}), numbering from about one hundred or more, to ranges of several thousand 1000 to 2500. Where the system is used in predicting epileptic seizure events, 60 minutes of EEG data is preferably used as initial baseline sample. Longer or shorter periods based on the type of data observed and a patent-based assessment based experience with the system may, however, be used. The initial time interval period (L) is preferably selected at about a first 20 minutes, with individual sampling time intervals preferably selected at about 20 seconds with the result that 180 data points (3 for every minute) are chosen.

An initial baseline data series is S_{N} (x₁, x₂, . . . x_{N}) is generated which preferably includes 60 minutes of EEG data containing 180 data points.

The selections of 60 minutes of data and the 20 minute L time interval are selected based on reasonable values that would fit the case being evaluated and do not constitute fixed values to be used in every application. Using the data points, “Fractal dimension”, P&H and “Lyapunov exponent” calculation are used to achieve a single constant that characterizes a non-linear data reference value of the initial interval time series V(S_{N}) for the monitored period.

The Data Series S_{N} is transformed to the new data series S^{m}_{N}={y_{L}, y_{L+1}, . . . , y_{N}} having new values “V”.

“Fractional Dimension” or “Lyapunov” or “P&H” calculate “a” value for data points (x₁, x₂, . . . x_{L}) initially for a first series (i.e. at time interval of 20 minutes) of data. This “V” value is chosen as the new data point/value y_{L}=V(x₁, x₂, . . . x_{L}) at time position “L” in the new transformed time series “S^{m}_{N}”. The data interval is next shifted one data interval or time series to series (x₂, x₃, . . . x_{L+1}), and the “V” value for this series is calculated which becomes the new data/point value at time position “y_{L+1}=V(x₂, x₃, . . . x_{L+1})”. The process repeats by the continued shifting of the time interval one data point position toward x_{N} and calculating “V” values which become the new data/point value at that time position until the x_{N} value has been transformed. This completes the data transformation to this point in time/data readings providing the new data series S^{m}_{N}

For the data series S^{m}_{N} created above (and which by way of non-limiting example runs from the L or 20 minute time point to the 60 minute time point and having 120 data points) calculate a “V” value using the same “Fractional Dimension” or “Lyapunov” or “P&H”. This value V(S^{m}_{N}) is then used as reference “V” value for predicting next future data point y_{N+i} values in the time series.

In the series y_{L}, to y_{N+1} (which in an exemplary application is selected from the 20 minute time mark to the 60 minute time mark) the value vertical difference between each consecutive data point y_{L} to y_{N} is taken and the normal distribution of these values N(yᵢ, σ²) is calculated. Using the calculated normal distribution N(yᵢ, σ²) above centered on the Y-axis of y_{N}, a series of new random data points or values are generated by a random number generator on the next time increment to be evaluated for the next point to be predicted on line at the time interval T_{N+1}. Preferably between 5 and 25 and about 10 random data points are generated following the normal distribution curve N, however fewer or larger numbers may be used.

For an associated data sequence containing each of the randomly generated points, generated by the random number generator on line y_{N+1}, a new “V” value is established using the data sequence (y_{L}, y_{L+1}, . . . y_{N}, y^{j}_{N+1}), where y^{j}_{N+1} represents the new points generated by the random number generator. As a result, where 10 random numbers are used Vj values separate V₁ to V₁₀ are calculated to develop V₁=V(yᵢ, yᵢ₊₁, . . . y_{N}, y¹_{N+1}), V₂=V(yᵢ, yᵢ₊₁, . . . y_{N}, y²_{N+1}), . . . V₁₀=V(yᵢ, yᵢ₊₁, . . . y^{N}, y¹⁰_{N+1}).

Each of the calculated Vj values are compared to the V(S^{m}_{N}) reference value, and the Vj value closest to V(S^{m}_{N}) is selected as the new predicted point y_{N+1}=y^{j}_{N+1}.

The steps of randomly generating data points and establishing and comparing calculated Vj values and reference values are repeated until preferably at least sixteen minutes of data, and more preferably approximately one-third of the reference data is projected into the future.

In one aspect, the present invention resides in a monitoring system for providing a user with advance warning of a likely seizure event, and wherein the system comprising: a signaling mechanism operable to provide at least one of an audible, visual or sensory warning signal to said user indicative of a predicted seizure event; a sensor assembly having at least one sensor and preferably a wireless microsensor operable to sense and output sensed data values representative of the user's electroencephalographic (EEG) wave forms readings substantially continuously, a computing device having a processor and memory, the computing device electronically, and preferably wirelessly, communicating with said sensor assembly for receiving the output sensed data in said memory, the processor including program instructions operable to perform one or more of process steps described hereafter.

In another aspect, the PDA operates to select a spot value from the continuous data streaming into the PDA at a constant or same set time interval, and which is determined by the characteristics of the input data. In the case of EEG data, a preferred data sampling time interval of 20 seconds was determined, with a baseline data recording time of 60 minutes of user EEG data being used. An initial data analysis window (L) of 20 minutes was also selected to provide a 15 minute prediction target which falls within the window. The selected baseline data points for the entire 60 minute baseline data series may thus be represented as:

S_{N}=(x₁,x₂, . . . x_{N})

[i.e. in this embodiment x_{N} is data point 180]

To predict future events from EEG data, a first subset of data over the first initial 20 minutes (S_{L}) is chosen, and the data is preferably first transformed into a transformed data set S^{m}_{N} that is used in the data prediction model. The data transformation developed for EEG data to create a transformed data set, where the predictive model described can be applied is outlined in accordance with the following process, and wherein

S_{L}=(x₁,x₂, . . . x_{L})

[in this example x_{L} is data point 60]

In a preferred operating mode in accordance with another aspect of the invention, the processor is operated to transform the collected EEG data value readings for the initial set sampling period S_{L}=(x₁, x₂, . . . x_{L}) for the data set over the selected recording period L to a single V value, V(S_{L}), using one or more of Fractional Dimension, Lyapunov, and P&H. In particular, V(S_{L}) is a measure of chaos in sample S_{L} and is plotted a new value V(S_{L})=y_{L} at time line point L. The data window “L” is then moved one data point right to form the new data set S(_{L+1})=(x₂, x₃, . . . x_{(L+1)}). A new V_{(S(L+1)}) is calculated as before, and the V(S_{(L+1)}) value is set y_{(L+1)} is plotted in the time line at position (L+1). This is then repeated until all the remaining EEG data points in the baseline data set S_{N} are converted, providing new transformed data set of transformed data points (y) from time point L to N. This then provides the new transformed value time series for time points “L” through “N” as follows”

S_{(LN)}=(y_{L},y_{L+1},y_{L+2}, . . . y_{N})

[in this embodiment S_{LN} contains 120 data points from data points 60 to 180]

The transformed value time series (S_{(LN)}) is then used as the data series upon which the predictive model can be applied.

Further each new EEG reading preferably goes through a corresponding transformation (from point x_{N+new} to y_{N+new}) before that data point is used as part of the time series upon which the predictive model is applied. The next EEG read data point therefore becomes x_{N+1} which is in turn transformed as above to become the new updated times series ending in data point y_{N+1} in the data series.

The prediction is carried out as follows on the new transformed data series S_{LN}.
A. For the initial transformed data series S^{m}_{N}, calculate a V reference value V(S^{m}_{N}) which is computed using as a reference value at least one of Fractal dimension or Lyapunov exponent or P&H. This will then become the V(S^{m}_{N}) that will be used to select predicted values.
B. The parameter σ of a normal distribution N(yᵢ,σ²) of the data series S^{m}_{N} is computed on the Y axis value, as differences between two consecutive points (y_{L}, y_{L+1}, y_{L+2}, . . . y_{N}) for all the points in the time series S^{m}_{N}.
C. To predict the next data point y_{N+i}, the normal distribution calculated in step B above N(y_{N+i-1},σ²) is centered on the Y axis value of the last point in the time series, y_{N}. Preferably at least five, and more preferably 10 or more random number values are generated within that normal distribution in time series position y_{N+i}. For each predicted point the same number of random numbers is preferably generated each time.

Pos(y_{N+i})={y^{j}_{N+i},1≦j≦N_{rand}.}
D. For each dataset containing each of the values generated by the random number generator S^{j}_{N+1} calculate a value V(S^{j}_{N+i}) using at least one of Fractal dimension or Lyapunov exponent or P&H.

S^{j}_{N+1}=S_{N+i-1}+y^{j}_{N+1}={y_{L},y_{L+1},y_{L+2}, . . . y_{N},y^{j}_{N+i}}
E. A next predicted data point y_{N+i} is selected as the V(S^{j}_{N+i}) value that is closest to V(S^{m}_{N}).

jₘᵢₙ=arg minⱼ(|V(S^{m}_{N+i-1+y}^{j}_{N+i})−V(S^{m}_{N})|)
F. Steps “C” and “D” are then repeated to predict future points of the time series, preferably creating a sequence of 60 predicted data points or with a 20 second sampling interval 20 minutes of prediction of the time series in this application.
G. An epileptic episode is predicted or likely determined if a selected number, and preferably 3 or more consecutive predicted values exceed the threshold value determined from the initial time series, and preferably may exceed about two standard deviations, and which in this example has a value of 2.4 or more. Suitable visual and/or audible or sensory warning signals are provided to the user and/or medical practitioners or other individuals/entities on the occurrence of such a prediction.
H. Each next data reading received from the EEG x_{N+1}, is then transformed by the described data transformation into new actual data point y_{N+1}. The data projection is then recalculated starting with new predicted point y_{N+1+i}, repeating steps “C” to “G”, creating a new projected series 20 minutes into the future. In calculation B. the time series used in this calculation most preferably starts at time point y_{L} and continues to grow in length as new actual data becomes available.

In another aspect the present invention resides in a seizure monitoring system having a signaling mechanism for providing a user with advance warning of a predicted epileptic seizure event, the system further comprising: a sensor assembly having a sensor operable to sense and output a plurality of data signal values representative of the user's electroencephalographic (EEG) activity over a monitored period of time as sensed data, a computing device having a processor and memory, the computing device electronically communicating with said sensor assembly for receiving the sensed data, the processor including program instructions operable to:

A. select and store in said memory said data signal values at a plurality of equally spaced time intervals over said monitored period as a first time series data sequence, and which for example maybe used to initiate the system and establish constants to be used in predicting future data points;

B. compute an initial base-line non-linear measure values to create an initial time series of data points (S_{N}) which are then transformed into a new data time series (S^{m}_{N}) sequence using fractal dimension, Lyapunov exponent and/or P&H.

The system is preferably operable to output by a signaling device an alarm and/or warning signal to warn the user of the likelihood of an epileptic seizure or other medical event. While the foregoing describes an initial 60 minute monitoring period, and a 20 second data sampling internal interval window L selected at 20 minutes, longer and shorter monitoring interval windows, and data sampling intervals may be used.

In a further aspect, the present invention resides in a method of using an EEG monitoring device and/or system for providing a user with advance warning of an epileptic seizure event, the system comprising: a sensor assembly having a sensor operable to sense and output a plurality of data signals representative of the user's electroencephalographic (EEG) activity, a computing device having a processor and memory, the computing device electronically communicating with said sensor assembly for receiving the sensed data. The sensor assembly is operable, sensing and continuously outputting to said computing device data signal of said user's EEG activity over an extended period of time. Preferably, data signal values sampled and recorded are stored in the memory plurality of equally spaced time intervals over said initial monitored period as an initial time series data sequence. Where values in the predicted time interval of the new time series data sequence has at least one, and preferably 3 or more consecutive values that exceed a preselected threshold value and preferably in the case of Tonic-clonic seizures, a standard deviation value of 2.4, output by the signaling mechanism a warning signal to the user indicative of the likelihood of said epileptic seizure event.

In one preferred aspect, the forward prediction limit is one-third the baseline recording period N/3, and preferably should be less than the time interval L and at least a period of about 16 minutes.

The applicant has appreciated by setting a predetermined threshold value of transformed EEG signal readings, the PDA may advantageously compare the data value which is projected to occur in the future with the predetermined threshold value. If the projected value equals or exceeds the predetermined value for a selected number (i.e. 3 to 6 and preferably about 3) consecutive points, the PDA operates to provide the user with an advance warning that an epileptic event is likely to occur. In such cases, the advance warning will occur less than the predicted time interval in the future. In a simplified mode of operation the PDA operates to provide an audible and/or visual warning to the wearer and/or a third party. In an alternate configuration, the PDA may be operable to provide the user with a physical warning signal, such as a vibration or sensory warning and/or a countdown clock, counting down the time remaining to the likely epileptic event, to better assist the wearer in curtailing potentially hazardous activities and/or allowing him or her to relocate to a safe environment.

In addition to the above discussed general aspects, the invention further provides for various preferred, non-limiting aspects, and which include:

1. A device, system and/or method in accordance with any of the aforementioned aspects wherein the processor is operable or further includes program instructions to change baseline reading interval; EEG electrode used for data source; data sampling interval; algorithm used to set V values; number of random numbers generated for predicting new values; restart system; etc.; with build in diagnostics of the system verifying that all aspects of the system are functioning properly and providing user a green light signal confirming same. In the event that the system encounters a functional problem the user is alerted both by visual signal and audible signal of system malfunction along with screen displays as to the nature of the malfunction.

2. A system and/or method in accordance with any of the aforementioned aspects wherein the fixed data sampling interval is consistent and is selected based on data being used and desired prediction period into the future. This may preferably range between about 1.0 second to hours and/or days.

3. A system and/or method in accordance with any of the aforementioned aspects wherein the analytic data window/interval is selected with sufficient data points to provide sample size of statistical significance based on data being analysed. In the case of EEG reading a preferred window of about 20±5 minutes containing 60 data points was selected.

4. A device, system and/or method in accordance with any of the aforementioned aspects wherein the base data set from which to make predictions of future data points is at least two times the data window, more preferably at least three times the data window.

5. A device, system and/or method in accordance with any of the aforementioned aspects wherein the plurality of random data values used to predict the next data point and greater than 4, and more preferably from about 10 to upto several thousand, depending on required accuracy.

6. A system and/or method in accordance with any of the aforementioned aspects further including a random number generator for generating the random data values in the value range calculated for the data centered on the value of the last point in the series.

7. A system and/or method in accordance with any of the aforementioned aspects wherein a threshold value is established above which epileptic seizures were most likely to take place is selected at between about 1.2 and 4, and preferably about 2.4; and/or preferably where 3 to 10, and preferably 3 or 4 consecutive points are calculated as falling above the threshold value.

8. A system and/or method in accordance with any of the aforementioned aspects wherein said output signals comprise EEG readings over a plurality of constant time intervals, and the initial time period is selected as a time period consisting of one or more pre-seizure, seizure and post seizure events.

9. A system and/or method in accordance with any of the aforementioned aspects wherein the computing device comprises a personal digital assistant, and said signaling mechanism comprises at least one of a visual display and an audio output, and wherein the output warning signal comprises at least one of an audible signal emitted by said audio output and a visual signal visible on said visual display. The system further may include various wearable electronic components, including micro adhesive attached sensors, smart watches and/or glasses, and the like.

10. A system and/or method in accordance with any of the aforementioned aspects wherein the seizure event comprises a Tonic-clonic seizure.

11. A system and/or method in accordance with any of the aforementioned aspect, wherein the processor is operable to provide the wearer with a visual and/or audible indication which indicates either a percentage or relative likelihood and/or severity of the project seizure event. In a more preferred mode, the PDA may be operable to provide different coloured graphic warning, where for example a red warning signal appears where there is a high probability of a severe seizure, or a yellow warning indicator is provided where the risk of a seizure is moderate, increasing and/or decreasing.

12. A system and/or method in accordance with any of the aforementioned aspects, wherein in the event the predicted future event is determined from EEG signals to represent a critical value, as for example if determined to deviate from one or more previously selected and/or averaged levels of chaos of EEG signals by a threshold amount, the system may be used to effect the display of a warning signal of a likelihood of a seizure or other pending events; and/or further transmit audible or electronic instructions to the user to cease any potentially hazardous activities such as driving or machinery operation, and/or to relocate to a safe environment.

### BRIEF DESCRIPTION OF THE DRAWINGS

Reference may be had with the following detailed description, taken together with the accompanying drawings, in which:

FIG. 1 shows schematically a system for predicting and outputting to a user an advance warning of a likely epileptic seizure in accordance with a preferred embodiment of the invention;

FIG. 2 illustrates graphically the user's recorded EEG data measured by the sensor of the system of FIG. 1 over a twenty minute monitoring and recording period, in accordance with the present invention;

FIG. 3 shows graphically the discretization of the recorded EEG measured data shown in FIG. 2 into a time series data sequence (x₁, x₂, x₃ . . . x_{N}) in accordance with the preferred embodiment of the invention;

FIG. 4 illustrates graphically the step of generating predicted next time data values used to effect timed series prediction to generate future predicted EEG values; and

FIG. 5 shows graphically the system output illustrating the predication of a future epileptic seizure, in accordance with the preferred embodiment of the invention.

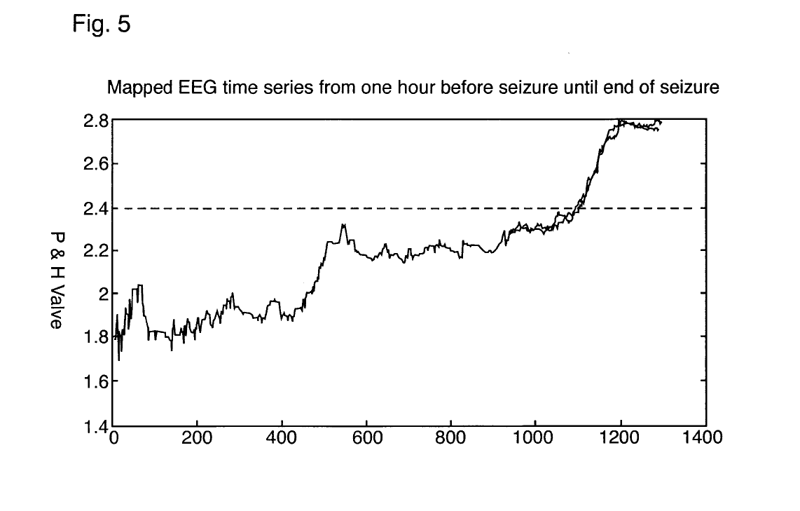

### DETAILED DESCRIPTION OF THE PREFERRED EMBODIMENTS

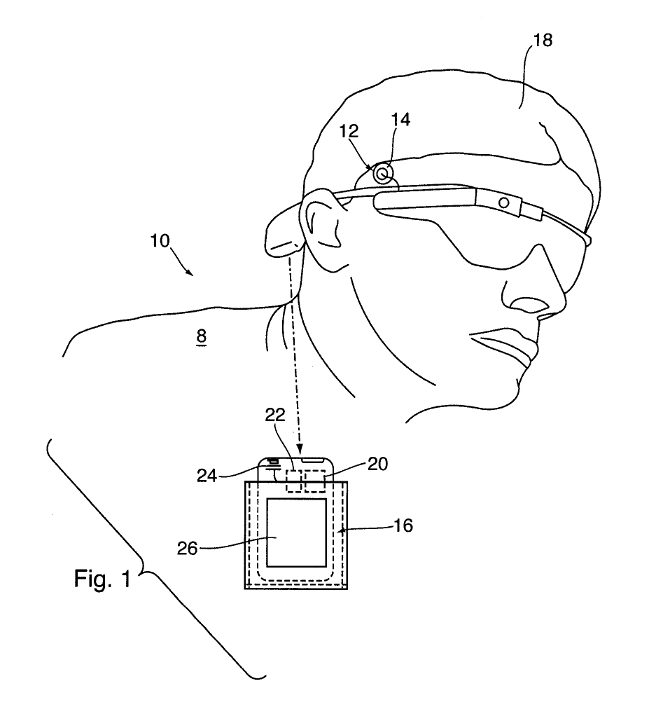

Reference may be had to FIG. 1 which illustrates a system 10 for use by a user 8 in predicting the likely occurrence of an epileptic seizure or event in accordance with a preferred embodiment of the invention. The system 10 includes a sensor assembly 12 having at least one electroencephalography (EEG) sensor 14 and personal digital assistant (PDA) 16.

As shown, the sensor 14 is adapted for placement in juxtaposed contact with a user's skull 18, and is operable to measure and record the electrical activity or electrical fluctuations of the user's brain. In one possible construction, the sensor assembly 12 may be provided as part of a smart glasses design, such as Google® glasses, or other such wearable technology. The sensor assembly 12 is operable to collect the EEG readings and wirelessly transmit them to the PDA 16 as a series of data readings or measurements taken over an initial sampling or monitoring period of from about ten to one-hundred and twenty minutes and preferably about twenty minutes. It is to be appreciated, that while FIG. 1 shows the system 10 as having a single EEG sensor 14, the invention could equally be provided with additional and/or different types of sensors 14 to detect and transmit to the PDA 16 more comprehensive data respecting the measured EEG readings.

The PDA 16 is provided with an internal antennae (not shown) adapted to wirelessly receive signals from the sensor assembly 12, and includes an internal memory 20, a processor 22, an audio output speaker 24 and a visual display screen 26. As will be described, the audio output speaker 24 and display screen 26 are operable in the use of the system 10 to provide the user 8 with both an audio and visual warning of a predicted likelihood of an impending epileptic seizure or event.

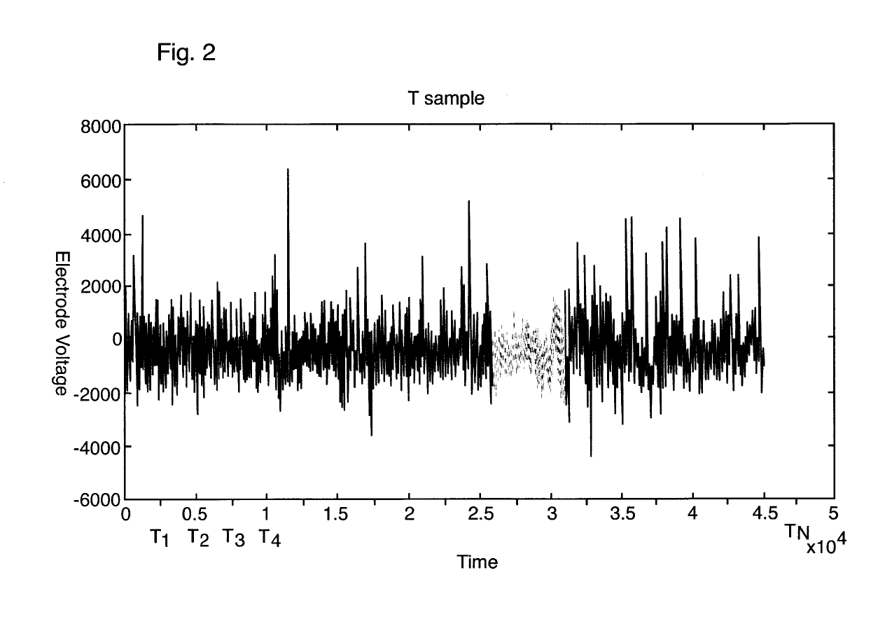

In a preferred mode of operation, the sensor 14 is operated to collect and transmit to the PDA 16 the user's EEG data over an initial sampling or monitoring period as time series Tₛₐₘₚₗₑ, where it is stored in the PDA memory 20 as measured relative EEG values. Preferably, EEG data is collected as a substantially continuous data file for the initial monitored period of time and thereafter, as for example is shown graphically in FIG. 2. More preferably, the monitoring period is chosen as a measured time period which is selected where the user 8 does not experience a seizure event such as a Tonic-clonic seizure, but also undergoes pre-seizure and/or post seizure EEG activities.

As will be described, the processor 22 includes programme instructions which are stored in memory, and which are operable to identify any measure the threshold value of transformed EEG signal values. The determined threshold value is then stored in the memory 20 as a preset threshold value which, based on the transformed historical data, provides a value above which is indicative of the occurrence a seizure event.

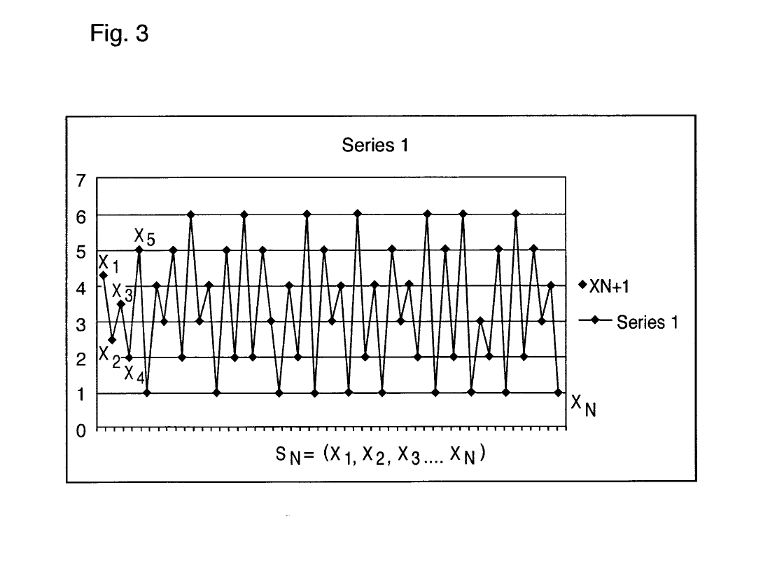

Once the initial monitored data is input and stored in memory 20, the processor 22 is used to transform the sensed data into a series of data values taken at equally spaced time intervals (i.e. preferably every twenty seconds). In particular, as shown in FIG. 3, the sensed data is used to generate a continuous baseline data sequence over period of 60 minutes, whereby data values are determined at each 20 second time interval (x₁, x₂, . . . x_{N}) over the selected monitored time interval, as shown in FIG. 3. In generating the initial time series data sequence shown:

1. The smallest time interval taken in the illustrated time series is the sampling time between x₁ and x₂, and the horizontal distance/time between points is always equal.

2. In the data time series shown graphically, the point on the left is x₁, with the final point or time interval on the right is x_{N} (x_{N} in the example shown in FIG. 3 is time sequence 180), with the subscription of each point increasing by 1 moving to x_{N}; the sequence of points (x₁, x₂, . . . x_{N}) in the initial measured time series data for the entire monitored 60 minute period may thus be expressed as S_{N} or S_{N}=(x₁, x₂, x_{N}).

Following the establishment of the initial measured time series data sequence S_{N}=(x₁, x₂ . . . x_{N}), over the sixty (60) minute period a first twenty (20) minute interval L of data (S_{L}) is chosen S_{L} (x₁, x₂, x₃ . . . x_{L}).

A non-linear measure value V(S_{L}) is then determined for the measured time series data sequence (S_{L}) as a reference value. Preferably, the processor 22 is used to calculate the non-linear reference value for the series S_{L} using one or more of “Fractal Dimension” or “Lyapunov” or “P&H”. The value V(S_{L}) represents the measure of chaos in sample S_{L} is plotted as a new value V(S_{L})=y_{L} at time point L. As such, for the first initial interval V(S₁)=y₁.

The data window L is then moved to the right one data point. A next time series for a next interval S_{L+1}=(x₂, x₃ . . . x_{L+1}) is then chosen and a non-linear measure V(S_{L+1}) computed using fractal dimension, the P&H value and/or Lyapunov exponent to generate a new V(S_{L+1}) [i.e. V(S₂)]y_{L+1} value [i.e. y₂].

The process is then repeated for all of the remaining “x” values in the measured time series S_{N} to generate a transformed data time series S_{LN} (y_{L}, y_{L+1} . . . y_{N}) For the initial transformed data series S^{m}_{N}, possible mapping may be required, forming the new time series S^{m}_{N}⁼{y_{L}, y_{L+1}, . . . y_{N}}:

yᵢ=V(S_{i-L+1,i}),L≦i≦N where S_{i-L+1,i}={y_{i-L+1},y_{i-L+2}, . . . y_{N}};

otherwise S^{m}_{N}=S_{N}

where 0<L<N is the size of a sliding window used to compute the local level of chaos measured by V( ). Therefore, when the mapping is applied, the new considered time series S^{m}_{N} corresponds to the variation in time of the local non-linear measure in the initial time series S_{N}.

V(S^{m}_{N}) is then determined as a reference value that will be used for predicting the next k values of the time series:

y_{N+i},1≦i≦k.

As will be described, based on the historical data collected during the initial 60 minute monitoring period Tₛₐₘₚₗₑ, the processor 22 is operable to read actual EEG then transform these values into values upon which future data values can be predicted. These predicted values may then be compared against the preselected threshold value to identify a likely epileptic event. In a simplified embodiment of the system 10, the PDA 16 outputs to the user 8 an alert signal or other identifier on the PDA speaker 24 and/or display 26, and preferably if three or more consecutive predicted future values exceed the preselected threshold value. Preferably, the PDA 16 is operable to provide a different warning or visual output signals to the user 8. Output warning signals may vary depending upon the resultant value of the predicted from the transformed EEG readings, and as it may relate to the probability of the seizure.

In a preferred operating mode, following the determination of the transformed time series S^{m}_{N}, a reference non-linear measure value of the transformed time series data V(S^{m}_{N}) is determined using Lyapunov exponent, P&H and/or fractural dimensions. The PDA processor 22 analyzes the transformed series S^{m}_{N}=y_{L}, y_{L+1}, . . . y_{N} taking the value difference between y_{L+1} and y_{L+2}, y_{L+3} and y_{L+4}, (FIG. 3), and so on to y_{N}, to calculate a normal distribution of the data series S^{m}_{N} on the Y-axis value N(yᵢ, σ²). Using the normal distribution of values at each time period N(yᵢ, σ²), a predicted next data point y_{N+} is calculated.

Preferably future time periods T_{L}, T_{L+1}, T_{L+2} . . . T_{N} are chosen as equal constant intervals of time (s) over the same selected duration of between 5 and 60 seconds, and preferably about 20 seconds.

The processor 22 operates to generate and output predicted future data values, at such time periods based on transformed data values that are used to create predicted values for the time interval (x_{N+1}) at point in time in the future, and preferably over a predicted future period of up to one third of the time covered by the measured baseline historical data points. Processor 22 performs a complex time series prediction based on an optimization process, whereby the processor 22 analyzes EEG data characteristics of the transferred time series S^{m}_{N}, and generates successively new predicted values y_{N+1} at successive points in time in the future, as continuing predicted time series. Further, as each new predicted data point is (y^{j}_{N+i}) generated, the processor 22 effects Lyapunov weighing and/or P&H methodology and/or fractal dimension to minimize the difference between the characteristic of the predicted new time series and the initial one.

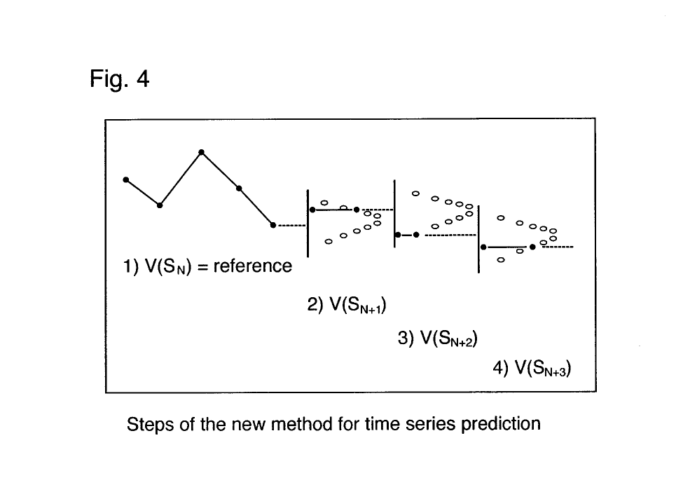

A most preferred method for long-term time series prediction is shown graphically in FIG. 4. As shown, using the normal distribution calculated for the transformed initial time series S_{N} described above, the distribution curve centered on the y_{N+i} 1≦i≦k value of the last data point of the time series data sequence.

In particular, the parameter a of the normal distribution N(yᵢ,σ²) 1≦i≦k of the transformed time series S^{m}_{N} is computed by computing the variation between every two consecutive values (i.e. yᵢ to yᵢ₊₁). This distribution represents the distribution of probability of value of yᵢ, knowing yᵢ₋₁ (FIG. 3).

Next, the processor 22 is used to generate randomly a number of potential predicted future values for the next time interval. The processor 22 preferably operates to generate, at least five to thirty, and preferably about ten new random values. In a simplified mode, random numbers are generated by way of a random number generator program for the next and as well be described, each subsequent time interval (y_{N+i+1}) to be evaluated at the next and each subsequent point to be predicted for time T_{N+1+1}. For predicting y_{N+i+1} Pos(y_{N+i+1}), each randomly generated valve of the set of r random values, are plotted following the normal distribution N(y_{N+i}, σ²) (FIG. 3). Therefore random r numbers are generated whereby:

Pos(y_{N+i+1})={y^{j}_{N+i+1}, 1≦j≦Nrand} is a parameter that can impact on the quality of the prediction, since having more values will increase the chance of finding an optimal value. However, it has been shown that in the analysis of EEG data significant improvement was not observed for the data when r was greater than ten.

For each of the random data values generated y^{j}_{N+i+1}, an associated extended generated time series is created (S^{m}_{N+i+1}=(y_{L}, y_{L+1} . . . , y_{N}, y_{N+1}, . . . , y^{j}_{N+i+1}). The extended generated time series sequence in then used to compute an associated non-linear measure value V(S^{m}_{N+i+1}) using fractal dimension, P&H method and/or Lyapunov exponent. As such, for each separate data set containing each ten predicted point generated by the random number generator, a new “V” (i.e. V₁, V₂ . . . V₁₀) value is established using the data sequence (y_{L}, y_{L+1} . . . y_{N+i}, y^{j}_{N+i+1}), where y^{j}_{N+i+1} is one of the r new points generated by the random number generator.

The generated time series sequence having the associated non-linear measure value (V₁, V₂, V₃ . . . V₁₀) closest to the reference value V(S^{m}_{N}) is then chosen as the predicted next time series data sequence S^{m}_{N+i+1}=(y_{L}, y_{L+1} . . . y_{N+i}, y_{N+i+1}). Further, the random data value y^{j}_{N+i+1} for the selected next time series data sequence is assigned as the predicted data value for the next time interval T_{N+i+1}.

y_{N+i+1} is thus computed by:

jₘᵢₙ=arg minⱼ(|V(S^{m}_{N+i-1}+y^{j}_{N+i})−V(S^{m}_{N})|) with (S^{m}_{N+i-1}+y^{j}_{N+i}={y₁,y₂, . . . ,y_{N+i-1},y^{j}_{N+1}})y_{N+1}=y^{jmin}_{N+i}

[y^{jmin}=minimum variance value between reference values and V values]

The value y^{j}_{N+i}ⁱ is chosen to make V(S^{m}_{N+i}+y^{j}_{N+i+1}) as close as possible to V(S^{m}_{N}).

### Test Data

Preliminary testing suggests that the present method and system may achieve a high degree of accuracy in providing epileptic patients with advance warning of the likely onset of a seizure.

In preliminary testing, 21 patients diagnosed with epilepsy were monitored. In particular, EEG (electroencephalography) data from each patient was acquired using a Neurofile NT™ digital video EEG system with 128 channels, 256 Hz sampling rate, and a 16 bit analogue-to-digital convert. For each of the patients, there were datasets celled “ictal” and “interictal”. As shown in FIG. 3, the EEG signal was discretized as a time series vector, X={x₁, x₂, . . . x_{N}} comprised of single electrical data readings at various time intervals and expressed as a series of individuals data points (single electrical readings by an electrode), where N is the total number of data points and the subscript indicates the time instant (FIG. 3). The P&H method was applied to the EEG time series to find the difference between seizure and seizure-free parts of EEG time series.

To evaluate performance of new method on prediction of epileptic seizure, the EEG time series measured by five electrodes, generating five different time series, for 21 patients were examined. For each EEG time series, the exact time of seizure was known and recorded. The P&H chaoticity values were predicted using GenericPred. The P&H chaoticity values were calculated on a constant-length (20 minutes) sliding window, with sliding time intervals of 20 seconds, of the EEG time series. During seizure, a peak in P&H values obtained from EEG time series appears. Based on the analysis of all 21 patients, a threshold for prediction of seizure determined at a preselected P&H value equal to 2.4 or greater (see FIG. 4) providing a reliable indication as a threshold EEG value indicative of the onset of a Tonic-clonic type seizure event.
a. In undertaking testing to determine the prediction of a likelihood of epileptic seizures, 60 minutes of EEG data was selected as an initial base. This was based on the type of data observed and a best assessment. As a result, 180 data points (1 for every twenty (20) seconds) were analyzed as the initial time series data sequence;
b. The time step or interval between data points/readings was chosen as a constant at 20 seconds;
c. EEG data was transformed, to permit it to be used for predicting into the future;
d. Time interval “L” was chosen at 20 minutes; and
e. The total data series chosen S_{N}=(x₁, x₂, . . . x_{N}) was 60 minutes of data containing 180 data points.

The selection of 60 minutes of baseline data and the 20 minute time interval L were selected based on an expectation of reasonable values that would fit the case being evaluated, and were not provided as fixed values to be used in every application. As such, larger or shorter baseline and/or time interval data may be used.
1. The initial data was transformed to provide new predictive data series S^{m}_{N}={y_{L}, y_{L+1}, . . . y_{N}} having new values as follows:
   i. Using “Fractal Dimension”, “Lyapunov” and/or “P&H”, calculate a “V” value for the data series S_{L} comprising data points (x₁, x₂, . . . x_{L}) over the first 20 minutes of data. This “V” value becomes the new data point/value y_{L}=v(x₁, x₂, . . . x_{L}) at time position “L” in the new transformed time series “S^{m}_{L}”
   ii. The 20 minute data interval was then shifted by one time interval to series (x₂, x₃, . . . x_{N+1}), and a next “V” value is calculated for the shifted interval series which becomes the new data/point value at time position “y_{L+1}=V(x₂, x₃, x_{N+1})”
   iii. The shifting of the 20 minute time interval is continued one data point or time interval position toward x_{N}, and subsequent “V” values are calculated which become the new data/point value at that time position until the x_{N} value has been transformed. This completes the data transformation to this point in time/data reading, providing an initial transformed data series S^{m}_{N}
2. For the initial transformed data series S^{m}_{N} created in 1 above (which runs from the L or 20 minute time point to the 60 minute time point and having 120 data points) calculate a “V” reference value using the same “Fractal Dimension”, “Lyapunov” and/or “P&H” as described above. This V(S^{m}_{N}) is stored as a “V” value used as a reference for predicting the next data point y_{N+1} values in the time series.
3. Now in the series S^{m}_{N} (which is from the 20 minute time mark to the 60 minute time mark) the value of the vertical difference between consecutive points y_{L}, y_{L+1}, y_{L+2} . . . y_{N} is taken for all points and the normal distribution of these values calculated N(yᵢ, σ²)
4. Using the normal distribution calculated, and centered on y_{N}, create multiple and preferably about ten new random values y^{j}_{N+i} which are generated by a random number generator at the next projected time increment to be evaluated, for the next point to be predicted on time T_{N+i} 1≦i≦k (and where for the first increment i=1)
5. For each of the new random values generated by the random number generator on time T_{N+i} establish a new “V” value as above using its data sequence S^{j}_{N+i}=(y_{L}, y_{L+1}, . . . y_{N}, y^{j}_{N+i}), where y^{j}_{N+i} is one of the 10 new points generated by the random number generator. As a result, (Vj) values V₁, V₂ . . . V₁₀ are developed as V₁=V(y_{L}, y_{L+1}, . . . y_{N+i}, y¹_{N+i}), V₂=V(y_{L}, y_{L+1}, . . . y_{N+i}, y²_{N+}), . . . V₁₀=V(y_{L}, y_{L+1}, . . . y_{N}, y¹⁰_{N+1}).
6. Each of the V₁ to V₁₀ values are then compared to the references V(S^{m}_{N}) value, for the initial time series and the Vj with the value closest to V(S^{m}_{N}) is selected as the predictive time series, and the associated randomly generated value y^{j}_{N+1} is chosen as the next new predicted point y_{N+i+1}=y^{j}_{N+i+1}.
7. Calculations #3 to #6 are then repeated to establish the next data value prediction at a next time interval using y_{N+i+1} instead of y_{N+i}. This is repeated until 20 minutes of data is projected into the future.
8. Next the calculation starts again after the next new raw data point x_{N+1} is received into the system.

It is to be appreciated that establishing “V” values using “Fractal Dimension”, Lyapunov” and/or “P&H” are based on what is more appropriate for the application. It may also be acceptable to calculate “V” values using a combination of values of two or more such methods (“Fractal Dimension”, “Lyapunov” or “P&H”).

Using the P&H threshold value, the current method was shown to predict future epileptic seizures with a high degree of sensitivity and specificity up to 17 minutes in advance (see Table 1 below). Further, different ranges of EEG time series were considered before and after seizure (we considered 10 ranges during seizure-free part of EEG time series for each patient) and there was no peak predicted by the current method in any case.

For each patient, one positive and 10 negative samples were constructed. The positive sample contains one epileptic seizure event, and the 10 negative samples are seizure-free. Therefore, there are 21 positive and 210 negative samples in total that were used to compute the specificity and the sensitivity accuracy levels.

TABLE 1
Sensitivity and specificity of epileptic seizure prediction
for 21 patients for different lengths of prediction.

| Length of prediction before seizure | Sensitivity | Specificity |
|---|---|---|
| 16 minutes ± 7 seconds | 100% | 100% |
| 17 minutes ± 7 seconds | 100% | 100% |
| 18 minutes ± 13 seconds | 85% | 100% |
| 19 minutes ± 13 seconds | 57% | 100% |
| 20 minutes ± 43 seconds | 43% | 100% |

The same results were obtained by considering the data of any five electrodes independently. This is believed to represent an improvement over other predictive method, which typically achieves accuracy levels of 73% sensitivity and 67% specificity for 10 patients within a 1-19 minute range.

It is not anticipated that the current method will provide 100% sensitivity and specificity in all instances. Preliminary testing has, however, suggested that the system and method of the present invention shows strong promise in providing a good indicator of the likelihood of the onset on an epileptic event.

### Further Applications

Although the detailed description describes the current method and system as most preferably being used for predicting epileptic seizures, the current system shows promise for a wide variety of different applications.

In an alternate mode, the method of the present invention may be used to predict the possible onset of a heart attack or stroke, as for example, by assessing chaotic variability of blood pressure changes, heart beat or heart arrhythmia. In yet another embodiment, the system 10 may be adapted for use as a medical warning device, as a predictor for the likelihood of the onset of seizure heart attack. The system 10 may include electro-cardiogram (ECG) in place of electroencephalogram (EEG) sensors to provide data representative of a patient's heart palpitations or arrhythmia over a historical or monitored time period.

In yet a further alternate possible application, the system 10 may be used as a predictor for future angina attacks. In particular, a patient's blood pressure data may be monitored over a selected period of time and input into the processor memory 20. By the aforementioned process, the processor 22 is activated to identify the future times where a potentially critical high blood pressure event is likely, and which correlates to a patient angina attack.

Again, on predicting the possible onset of such an occurrence, the system 10 could be used to provide either visual or audible warning to a user or medical practioner via the display 26. Alternately, if provided as part of an automatic drug dispensation system, the processor 22 could be used to output control signals to effect an adjustment of a pacemaker or an automated drug dispensation apparatus to alter the medical dosage of a patient's heart medication in anticipation of the possible angina event.

In a further non-limiting embodiment, the system 10 may be used to establish predictive environmental models. In one embodiment, data representing past measured amounts of vegetative growth of a particular plant or algae may be input for a selected historical time period. Using the foregoing method, the processor 22 may provide output data which is predictive of when a selected plant species may dominate or be subordinated relative to other species within a particular geographic area.

It is noted that establishing “V” values using “Fractional Dimension”, “Lyapunov” or “P&H” are based on what is more appropriate for the application. It may also be acceptable to calculate “V” values using the averaged values of one, two or all of these methods.

Each of the new non-linear data values (V₁, V₂ . . . V_{N}) are compared with the earlier calculated (V^{m}_{N}) reference value, and generated time series with the closest corresponding value is selected, with its associated random data value chosen as the prediction for the next predicted time interval value in the time sequence. Using the generated time series sequence, the next subsequent predicted data value is determined by repeating steps of randomly generating and selecting data points by their approximation to the initial reference value. The process calculations may continue to be used to generate new predicted data values or points. Most preferably, number of new data points created in the sequence does not exceed one third of the total number of historic data points (N/3).

As a result, with the present method historical data may be rapidly updated. Most preferably, instead of making a shift of N data points at a time, a shift of a single data point is undertaken. That means that just one new real point value is measured (N+1) and then the new historical data to be taken into account are (2, 3, . . . , N+1), and the new prediction begin at N+2.

In the preferred mode, the reference value is always V(S^{m}_{N}), and which is obtained based on the value of the transformed non-linear data series from the original time interval. Therefore, with to the present method it is advantageous to keep the value of a transformed non-linear measure steady as much as possible during prediction (see FIG. 3). As used, the new predicted value is chose from a set of potential values generated from a distribution of probability in an acceptable selection range.

With the current system 10, prediction is performed using the complete time series whereas, in traditional approaches, after computation of the model, prediction is performed only using the model and no longer the original time series. Therefore, the current model allows for constant adjustment of information about the current time series, whereas classical predictive methods apply the model without taking into account the accordance between the original time series properties and the predicted ones. Moreover, the optimization step allows making choice among a set a potentially good predictive values, compared to the traditional models which only generate one value. Another advantage of the present invention is that it does not rely on a complex model of the original time series and it is therefore very general. Having no specialized model for prediction makes new method less restricted to a specific domain.

The system 10 most preferably incorporates built-in diagnostics software operable to verify that all aspects of the system 10 are functioning properly, and outputting via the display a green light signal confirming same. In the event that the system 10 encounters a functional problem, the user is alerted both by visual signal and audible signal of system malfunction, along with screen display as to the nature of the malfunction.

The present method shows a strong improvement compared to traditional methods over different situations and other chaotic time series in term of accuracy both for short and long term prediction. Moreover, the present method shows ability to predict the trend of evolution of other chaotic time series is much better than those of existing methods. Its performances are also more stable, with a standard deviation of the error measure appearing lower than those of the other methods. The method provides step toward an accurate and comprehensive time series long-term prediction.

It should be noted that preferred embodiment of the present method is not customized for a specific application, using a similar non-linear criterion may have the same function for a variety of applications. Further, by involving knowledge from other fields, it may be possible to provide a universal method for predicting a variety of non-linear time series. In another embodiment, the present method could utilize several non-linear measures simultaneously, instead of using just one measure, to identify and preserve the complexity of time series more efficiently.

Although the preferred embodiment describes the system and process for use in the predictive analysis of epileptic seizures, it is to be appreciated that the present process and system is equally applicable across a number of other possible applications. Such applications could include without restriction, applications in predicting macrogeographic events and trends; the predictive modeling of pandemics and pathogenic outbreaks; weather and meteorological modeling; and/or earthquake and geological event modeling. In addition, the system and method may further be used in the prediction and/or analysis of other complex data of non-linear events, including heart attack and/or stroke, as well as part of a health monitoring or warning system to provide an advance indication of other types of likely health events.

Although the disclosure describes and illustrates various preferred embodiments, the invention is not so limited. Many modifications and variations will now occur to persons skilled in the art. For a definition of the invention, reference may be had to the appended claims.

1. A monitoring system for providing a user with advance warning of a likely seizure event, the system comprising:
a signaling mechanism operable to provide at least one of an audible, visual or sensory warning signal to said user indicative of a predicted seizure event;
a sensor assembly having at least one sensor operable to sense and output sensed signals representative of the user's electroencephalographic (EEG) wave forms over a baseline monitoring period,
a computing device having a processor and memory, the computing device electronically communicating with said sensor assembly for receiving the output sensed signals at selected time intervals over said baseline monitoring period,
storing in said memory sensed data values representative of said output sensed signals at spaced time intervals (x₁, x₂ . . . xₙ) over said baseline monitoring period,
the processor including program instructions operable to perform the process steps of:
A. select an initial data series sequence comprising associated one of said sensed data values over a selected timeline period comprising part of said baseline monitoring period, wherein said initial data series sequence is represented as
S_{L}={x₁,x₂ . . . x_{L}}
B. compute a first non-linear measure value V(S_{L})=_{L} of said initial data series sequence using at least one of fractal dimension, Lyapunov exponent and P&H;
C. for each subsequent remaining sensed data value over the baseline monitoring period S_{L+1}={x₁, x₂ . . . x_{L+1}} compute an associated non-linear measure values using at least one of fractal dimension, Lyapunov exponent and P&H to provide a transformed data series,
(S^{m}_{N})=(y_{L},y_{L+1},y_{L+2} . . . y_{N});
D. for the transformed data series sequence S^{m}(_{L,N}) calculate a reference non-linear value V(S^{m}_{N}) of the transformed data series sequence (S^{m}_{N}) using at least one of fractal dimension, Lyapunov exponent and P&H;
E. determine a normal distribution curve of the Y values between sequential data points (y_{L}, y_{L+1}, y_{L+2} . . . y_{N}) in the transformed value data series sequence S^{m}_{N};
F. with said normal distribution curve centered on the last data value y_{N} in said transformed data series (S^{m}_{N}), generate a plurality of random next data values (y^{j}_{N+1}) for a predicted next time interval (xₙ₊₁), as separate generated extended time series sequences (S^{j}_{N+1});
G. for each said generated extended time series sequence S^{j}_{N+1}, compute an associated non-linear measure value V(S^{j}_{N+1}) using at least one of fractal dimension, Lyapunov exponent or P&H;
H. select the generated extended time series sequence having the associated non-linear measure value (V(S^{j}_{N+1})) closest to the stored reference non-linear measure value V(S^{m}_{N}) as a next time series data sequence, and assigning the random data value for the selected extended time series sequence as a predicted data value (y_{N+1}) for the predicted next time interval (x_{N+1});
I. compare the predicted data value of the predicted next time interval with a predetermined threshold indicative of a likelihood of said seizure event, and wherein when at least one predicted data value exceeds a predetermined threshold, the computing device activating said signaling mechanism to output said warning signal to the user.

2. The monitoring system as claimed in claim 1, wherein the predicted data value is selected as a new last data value for an extended transformed data series, the processor further including program instructions to:
repeat steps F through I to generate successive predicted data values for next time intervals of at least about ⅓ of the time of the baseline monitoring period, and preferably at least about sixteen minutes.

3. The monitoring system as claimed in claim 1, wherein the selected timeline period is selected at from about a first one quarter to about one half of the baseline monitoring period, and preferably at about a first one third of the baseline monitoring period.

4. The monitoring system as claimed in claim 2, wherein the baseline monitoring period is selected at between about 45 and 120 minutes and the selected timeline period is selected at between about 10 and 30 minutes.

5. The monitoring system as claimed in claim 1, wherein the time intervals comprise equally spaced time intervals over the baseline monitoring period selected at between about 90 and 240.

6. The monitoring system as claimed in claim 1, wherein the plurality of random next data values is selected at between about 5 and 50.

7. The monitoring system as claimed in claim 6, wherein the system further includes a random number generator for generating the random data values.

8. The system as claimed in claim 1, wherein the threshold is selected as a standard deviation greater than about two to about 15.

9. The system as claimed in claim 8, wherein the spaced time intervals comprise equally spaced intervals selected at between about 1 and 120 seconds, and
the selected timeline period is selected at between about 15 and 30 minutes.

10. (canceled)

11. The system as claimed in claim 1, wherein said sensor assembly includes a plurality of said sensors, the output sensed signals comprising continuous electronic readings sampled over a plurality of constant time intervals, and the baseline period is selected as a time period consisting of at least two user experienced pre-seizure, seizure and post-seizure events.

12. The system as claimed in claim 1, wherein the computing device comprises a personal digital assistant, and said signaling mechanism comprises at least one of a visual display and an audio output, and wherein the warning signal comprises at least one of an audible signal to said user emitted by said audio output and a visual to said user signal visible on said visual display.

13. The system as claimed in claim 1, wherein the seizure event comprises a Tonic-clonic seizure.

14. A seizure monitoring system having a signaling mechanism for providing a user with advance warning of a predicted epileptic seizure event, the system comprising:
a sensor assembly having a sensor operable to sense and output sensed signals representative of the user's electroencephalographic (EEG) activity over a monitored baseline period of time as sensed data,
a computing device having a processor and memory, the computing device electronically communicating with said sensor assembly for receiving said sensed data, and operable to store in said memory a baseline time series comprising sensed data values (x₁, x₂, x₃ . . . x_{N}) representative of said sensed data at selected time intervals over the baseline period of time,
the processor including program instructions operable to:
A. compile an initial time series data sequence S_{L}=(x₁, x₂, x₃ . . . x_{L}) comprising data values over an initial recording portion of said baseline period of time;
B. compute an initial non-linear measure value V(S_{L})=y_{L} of the initial time series data sequence using fractal dimension, Lyapunov exponent and/or P&H,
C. compute successively, a non-linear measure value V(S_{L+1}) . . . V(S_{N}) for a time series data sequences comprising each subsequent data value in the baseline time series using fractal dimension, Lyapunov exponent and/or P&H as successive non-linear measure values [V(S_{L+1})=y_{L+1}] . . . [V(S_{N})=y_{N}];
D. store the non-linear measure values as a transformed value data series S^{m}_{N}, S^{m}_{N}=(y₁, y₂ . . . y_{N}) and determining a non-linear measure value for the transformed value V(S^{m}_{N}) data series S^{m}_{N} as a reference value;
E. determine a normal distribution curve of the non-linear measure values in the transformed value data series S^{m}_{N}; and
F. with the normal distribution curve centered on a last transformed value y_{N} thereof, generate from 5 to 50, and preferably about 10 random data signal values (y¹, y² . . . y^{N}) for a predicted next time interval (x_{N+1}), as part of an associated generated extended time series sequence S^{j}_{N+1};
G. for each said generated extended time series sequence (S^{j}_{N+1}), compute an associated non-linear measure value (V(S^{j}_{N+1})), using at least one of fractal dimension, Lyapunov exponent and P&H; and
H. select the generated extended time series sequence having the associated non-linear measure value (V(S^{j}_{N+1})) which is closest to the reference value V(S^{m}_{N}) as a new time series data sequence; wherein the random data signal value of the selected generated extended time series is selected as the predicted next data value y_{N+1} for the next projected time interval x_{N+1}, and
I. when at least one predicted next data value exceeds a preselected threshold value by a preselected amount, the system being operable to output by the signaling mechanism a warning signal to the user indicative of the likelihood of a future onset of said seizure event.

15. The system as claimed in claim 14, wherein the system is operable to output said warning signal when at least three successive predicted data values differ from said threshold value.

16. The monitoring system as claimed in claim 14, wherein following the selection of the predicted next data value, selecting the predicted next data value as a new last transformed value y_{N} associated with a new last time interval, the processor further including program instructions to:
J. repeat steps F to I.

17. The monitoring system as claimed in claim 14, wherein the baseline period of time is selected at about 60 minutes, and
the number of selected time intervals is selected at between about 150 to 200.

18. (canceled)

19. (canceled)

20. The system as claimed in claim 14, wherein the preselected threshold amount comprises a standard deviation of about 2.4±0.3.

21. The system as claimed in claim 14, wherein the baseline time series data sequence is divided into a plurality of said equally spaced time intervals, said initial time intervals being selected at between about 1 and 120 seconds.

22. The system as claimed in claim 14, wherein the initial recording portion of said baseline period of time selected at between about a first 10 and 30 minutes,
said data signal values comprise electronic readings over a plurality of constant time intervals, and the baseline period of time is selected as a time period consisting of two or more of a user pre-seizure, a seizure and a user post-seizure event.

23. (canceled)

24. (canceled)

25. The system as claimed in claim 14, wherein the epileptic seizure event comprises a Tonic-clonic seizure, and
wherein step J is performed to generate predicated next data values at next time intervals for a period of upto about ⅓ of the baseline period of time, and preferably is performed for at least about sixteen minutes.

26. (canceled)

27. An epileptic seizure monitoring and warning system for providing a user with advance warning of a likely seizure event, the system comprising:
a signaling mechanism operable to provide a warning signal to said user indicative of a predicted epileptic seizure;
a sensor assembly having at least one sensor operable to sense and output user sensed electroencephalographic (EEG) wave forms over an initial monitoring period,
a computing device having a processor and memory, the computing device electronically communicating with said sensor assembly and operable to receive the output sensed signals over said initial monitoring period, and store in said memory sensed data values representative of said output wave forms at approximately equally spaced time intervals (x₁, x₂ . . . x_{N}) over said initial monitoring period,
the processor including stored program instructions operable to perform the process steps of:
A. compile from said stored data values an initial data series sequence comprising associated ones of said sensed data values over a first timeline period, the first timeline period comprising between about 25% to 50%, and preferably about 33.3% of said initial monitoring period, wherein said initial data series sequence being represented as
S_{L}={x₁,x₂ . . . x_{L}}
B. compute a first non-linear measure value V(S_{L})=y_{L} of said initial data series sequence using at least one of fractal dimension, Lyapunov exponent and P&H;
C. for a next and each subsequent remaining sensed data value over a remainder of the baseline monitoring period, compute an associated non-linear measure value (y_{L+1}, y_{L+2} . . . y_{N}) using at least one of fractal dimension, Lyapunov exponent and P&H, to form a transformed data series sequence,
(S^{m}_{N})y_{L},y_{L+1},y_{L+2} . . . y_{N});
D. for the transformed data series sequence S^{m}_{N}, calculate a reference non-linear value V(S^{m}_{N}) of the transformed data series sequence (S^{m}_{L}) using at least one of fractal dimension, Lyapunov exponent and P&H;
E. determine a normal distribution curve of the Y values between each adjacent data point (y_{L}, y_{L+1}, y_{L+2} . . . y_{N}) in the transformed value data series sequence S^{m}_{N};
F. with said normal distribution curve centered on the last data value y_{N}, in said transformed data series (S^{m}_{N}), generate randomly a plurality of possible next data values (y^{j}_{N+1}) for a predicted next time interval (x_{N+1}), as part of a separate generated extended time series sequences (S^{j}_{N+1});
G. for each said generated extended time series sequence S^{j}_{N+}, compute an associated non-linear measure value V(S^{j}_{N+1}) using at least one of fractal dimension, Lyapunov exponent or P&H;
H. select the generated extended time series sequence having the associated non-linear measure value (V(S^{j}_{N+i})) closest to the stored reference non-linear measure value V(S^{m}_{N}) as a next time series data sequence, and assigning the next data value for the selected extended time series sequence as a predicted data value (y_{N+1}) for the predicted next time interval (x_{N+1});
I. compare the predicted data value of the predicted next time interval with a predetermined threshold value indicative of said epileptic seizure, and
J. repeat steps F through I for the selected extended time series data sequences, wherein the predicted data value of the selected extended time series data sequence is selected as a new last data value; and
wherein when at least two consecutive said predicted data values exceed the predetermined threshold, the computing device activating said signaling mechanism to output said warning signal to the user

28. The monitoring system as claimed in claim 27, wherein the initial monitoring is selected at about 60 minutes, and the time intervals are selected at between about 10 and 60.

29. The monitoring system as claimed in claim 27, wherein step J is performed to generate predicted next data value at next time intervals of upto about one third the initial monitoring period.
